(19) United States
(12) Patent Application Publication
(10) Pub. No.: US 2023/0346203 A1
(43) Pub. Date: Nov. 2, 2023

(54) ENDOSCOPE

(71) Applicant: OLYMPUS MEDICAL SYSTEMS CORP., Tokyo (JP)

(72) Inventors: Koichi TSURUTA, Yokohama-shi (JP); Hidenosuke HASE, Tokyo (JP)

(73) Assignee: OLYMPUS MEDICAL SYSTEMS CORP., Tokyo (JP)

(21) Appl. No.: 18/137,489

(22) Filed: Apr. 21, 2023

Related U.S. Application Data
(60) Provisional application No. 63/335,263, filed on Apr. 27, 2022.

(51) Int. Cl. A61B 1/00; A61B 1/015; A61B 1/12; A61B 1/018

(52) U.S. Cl. CPC A61B 1/00098; A61B 1/015; A61B 1/121; A61B 1/00103; A61B 1/018; A61B 1/00042

(57) ABSTRACT

An endoscope includes an insertion portion, an operation portion, a moving member, a detent, a rod, a wire and a bearing. The insertion portion is configured for insertion into a subject, the insertion portion including an actuating mechanism. The operation portion, the moving member, the rod and the wire are translatable in conjunction with each other. The detent located distally relative to the moving member, and the detent is configured to prevent a distally forward translation of the moving member past the detent. The bearing is located distally relative to the moving member, the bearing including an opening having a contact surface. The rod extends through the opening and is in slidable contact with the contact surface, and the contact surface is made of resin.

### RELATED APPLICATION DATA

This application is based on and claims priority under 37 U.S.C. § 119 to U.S. Provisional Application No. 63/335,263 filed on Apr. 27, 2022, the entire contents of which are incorporated herein by reference.

### FIELD OF DISCLOSURE

The present disclosure relates to an endoscope in which operation of an operating lever of an operation portion is mechanically transmitted to an actuating mechanism at a distal end portion of an insertion portion by a transmission mechanism.

### BACKGROUND

An endoscope is used to observe regions that cannot be seen from the outside by inserting an elongated insertion portion inside a subject. Also, a treatment instrument is inserted into a treatment instrument channel of the insertion portion, and a treatment is performed with the treatment instrument such as forceps protruding from an opening of a distal end portion. The insertion portion has a distal end portion at which a forceps raising base (forceps elevator) is disposed for directing the treatment instrument toward the affected area.

Japanese Patent Application Laid-Open Publication No. 2020-89598 discloses a medical apparatus configured to reduce friction generated by force acting in a bending direction with respect to an actuating direction in a transmission mechanism configured to transmit operation of an operating lever in an operation portion to the forceps raising base.

### SUMMARY OF THE DISCLOSURE

An endoscope according to an embodiment includes: an insertion portion, an operation portion, a moving member, a detent, a rod, a wire and a bearing. The insertion portion is configured for insertion into a subject, the insertion portion includes an actuating mechanism. The operation portion is disposed proximally relative to the insertion portion, and the operation portion includes an operating lever movable between a first position and a second position. The moving member is operatively connected to the operating lever, and the moving member is translatable between a proximal position and a distal position in response to an operation force from the operating lever. The detent is located distally relative to the moving member, and the detent is configured to prevent a distally forward translation of the moving member past the detent. The rod has a proximal end and a distal end, the proximal end of the rod is connected to the moving member and the rod is translatable in conjunction with the moving member. The wire is attached to the distal end of the rod, the wire extends into the insertion portion and is attached to the actuating mechanism, the wire is movable in conjunction with the rod to operate the actuating mechanism. The bearing is located distally relative to the moving member, the bearing includes an opening having a contact surface. The rod extends through the opening and is in slidable contact with the contact surface, and the contact surface is made of resin.

### BRIEF DESCRIPTION OF THE DRAWINGS

FIG. 1 is a perspective view of an endoscope system including an endoscope according to an embodiment.

FIG. 2 is a perspective view of a distal end portion of the endoscope of the embodiment.

FIG. 3 is a cross-sectional view of the distal end portion of the endoscope of the embodiment.

FIG. 4 is a configuration diagram of a transmission mechanism of an endoscope of a first embodiment.

FIG. 5 is a cross-sectional view of the transmission mechanism of the endoscope of the first embodiment.

FIG. 6 is an enlarged cross-sectional view of the transmission mechanism of the endoscope of the first embodiment.

FIG. 7A is a cross-sectional view of a fixing member in a modification of the endoscope of the first embodiment.

FIG. 7B is a cross-sectional view of a fixing member in a modification of the endoscope of the first embodiment.

FIG. 8A is a perspective view of a bearing of the endoscope of the first embodiment.

FIG. 8B is a perspective view of a bearing in a modification of the endoscope of the first embodiment.

FIG. 8C is a perspective view of a bearing in a modification of the endoscope of the first embodiment.

FIG. 8D is a perspective view of a bearing in a modification of the endoscope of the first embodiment.

FIG. 9A is a cross-sectional view of a bearing and a rod of the endoscope of the first embodiment.

FIG. 9B is a cross-sectional view of a bearing and a rod in a modification of the endoscope of the first embodiment.

FIG. 9C is a cross-sectional view of a bearing and a rod in a modification of the endoscope of the first embodiment.

FIG. 10 is a cross-sectional view of a moving member and a rail member of the endoscope of the first embodiment.

FIG. 11 is a cross-sectional view of a guide plate and a moving member of an endoscope of a second embodiment.

FIG. 12 is a perspective view of a stopping member having a guide plate of the endoscope of the second embodiment.

FIG. 13 is a perspective view of a moving member having a guided member of the endoscope of the second embodiment.

FIG. 14 is a cross-sectional view of a guide plate and a moving member of the endoscope of the second embodiment.

FIG. 15A is a perspective view of a stopping member having a guide plate in a modification of the endoscope of the second embodiment.

FIG. 15B is a perspective view of a stopping member having a guide plate in a modification of the endoscope of the second embodiment.

FIG. 16 is a partial cross-sectional view for describing a fixing member of an endoscope of a third embodiment.

FIG. 17 is a perspective view of a fixing member of the endoscope of the third embodiment.

FIG. 18 is a cross-sectional view of a transmission mechanism of an endoscope of a fourth embodiment.

### DETAILED DESCRIPTION

### First Embodiment

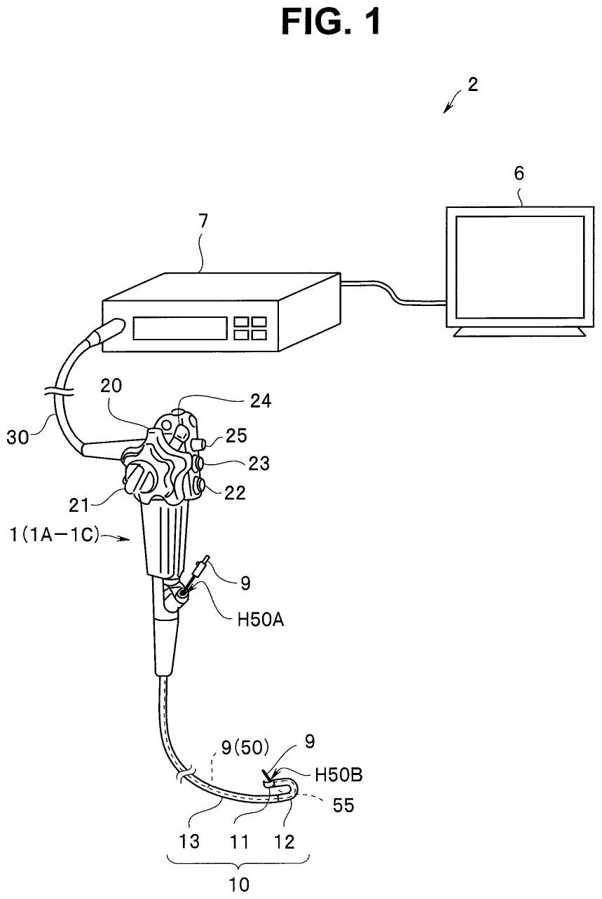

An endoscope 1 of an embodiment shown in FIG. 1 configures an endoscope system 2 together with a treatment instrument 9, a monitor 6, and a video processor 7. The endoscope 1 is, for example, a side-viewing duodenoscope to be used for treatment to take out gallstones from the common bile duct. The endoscope 1 is a single-use endoscope that is disposed of after a single use.

Drawings based on the embodiments are schematic. In the drawings, the relationship between the thickness and width of each portion, the ratio of the thickness of each portion, and the like are different from the actual ones. Among the drawings, there are also portions with different dimensional relationships and ratios from each other. Illustrations, and reference numerals and characters of some components are omitted.

The endoscope 1 includes an insertion portion 10 to be inserted into the body of the subject, an operation portion 20 provided proximally relative to the insertion portion 10, and a universal cord 30 extending from the operation portion 20.

The insertion portion 10 is provided, in the following order, with a distal end portion 11 on the distal end side, a bending portion 12 disposed on the proximal end side of the distal end portion 11, an elongated soft tube 13 connecting the proximal end side of the bending portion 12 and the operation portion 20. The distal end portion 11 has a forceps raising base (hereinafter referred to as a “raising base”) 55 that is an actuating mechanism to be described below.

The operation portion 20 is provided with a bending operation dial 21, an air/water feeding button 22, a suction button 23, an operating lever (forceps lever) 24 configured to operate the raising base 55, a plurality of operation switches 25. The bending portion 12 bends according to a turn of the bending operation dial 21. The operation switch 25 is an electronic switch configured to operate an image pickup unit (not shown) and the like.

The operation portion 20 is provided with an insertion opening H50A for introducing the treatment instrument 9 into the insertion portion 10. The insertion opening H50A connects to the proximal end side of a treatment instrument channel 50 inserted through the insertion portion 10. The treatment instrument channel 50 connects to an opening H50B of the distal end portion 11 of the insertion portion 10.

The video processor 7 including a CPU processes image pickup signals outputted by the image pickup unit and transmits image signals to the monitor 6. The monitor 6 displays an endoscopic image. The video processor 7 includes a light source circuit configured to generate illumination light and the like. The light source circuit may be included in a light source unit separate from the video processor 7.

### Configuration of Distal End Portion

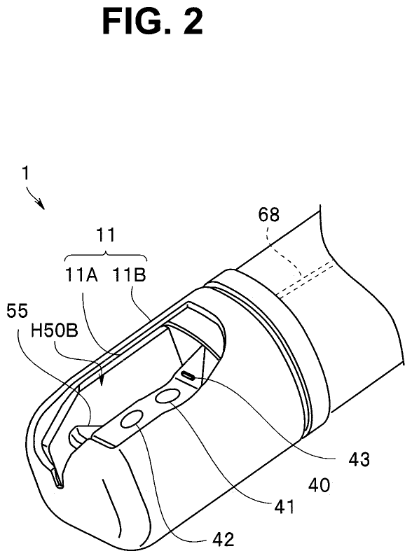

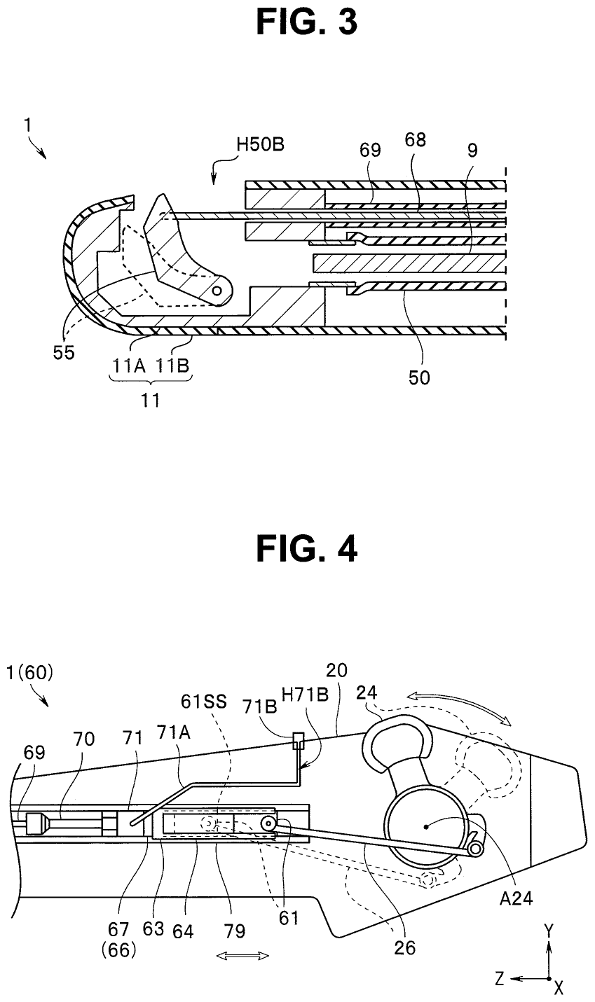

The distal end portion 11 of the endoscope 1 has a distal end component 11A, as shown in FIGS. 2 and 3, which is a rigid member made of metal or resin. A part of the distal end component 11A is covered with a distal end cover 11B. The distal end component 11A has an opening H50B inside which the raising base 55 is accommodated. The opening H50B is a space between a pair of arms of the distal end component 11A protruding toward the distal end side.

The raising base 55 is a tongue-shaped member. The treatment instrument 9 such as forceps is inserted into the treatment instrument channel 50 from the insertion opening H50A, and inserted through the treatment instrument channel 50. The treatment instrument 9 has a distal end effector for treatment that protrudes from the opening H50B of the distal end portion 11.

A direction in which the treatment instrument 9 protrudes from the opening H50B is operated by the raising base 55 that is the actuating mechanism. The raising base 55 is connected to the distal end of an operating wire 68. The raising base 55 turns in accordance with backward movement of the operating wire 68 to move from a lying state to a rising state. A protruding direction (protruding angle) of the treatment instrument 9 changes according to a rising angle of the raising base 55.

The insertion portion 10 is configured for insertion into the subject, the insertion portion 10 includes an actuating mechanism.

### Configuration of Transmission Mechanism

As shown in FIGS. 4, 5 and 6, a transmission mechanism 60 includes a moving member (piston rod) 61, rail members (guide member) 79 (79A and 79B), a stopping member (detent) 63 (79), a rod 65, a bearing (bush) 66, and an operating wire 68.

The transmission mechanism 60 further includes a wire pipe 69, a tubular member 70, a holding member (housing) 71, a first ring receiver 72, a first resin ring (first sealing ring) 73, and a second resin ring (second sealing ring) 74.

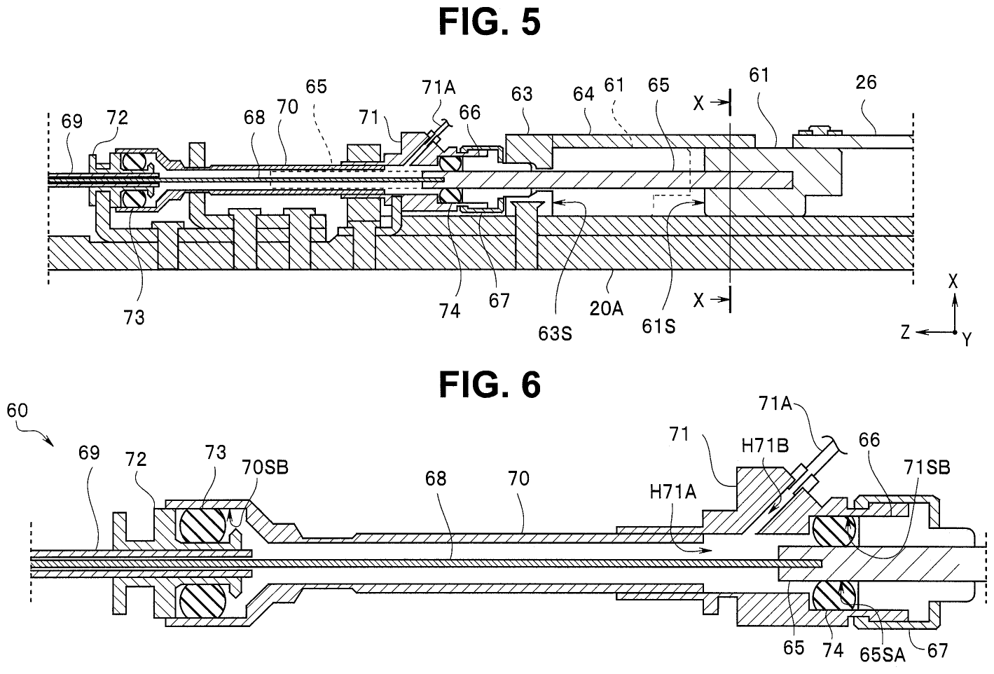

The moving member 61 sandwiched between the two rail members 79 linearly reciprocates in accordance with operation of an operating lever 24. In other words, the two rail members 79 sandwich two side surfaces 61SS parallel to a moving direction of the moving member 61. The stopping member 63 restricts a moving range of the moving member 61. For example, in FIG. 5, when a side surface 61S of the moving member 61 moves to a position indicated by a dashed line, the side surface 61S comes into contact with a side surface 63S of the stopping member 63 and stops. A rod 65 is a pulling member, the proximal end side of which is connected to the moving member 61, and the rod 65 can be made of metal.

The moving member 61 is operatively connected to an operating lever 24, the moving member 61 is translatable between a proximal position and a distal position in response to an operation force from the operating lever 24.

The guide member 79 is located adjacent to the moving member 61.

The detent 63 is located distally relative to the moving member, the detent 63 is configured to prevent a distally forward translation of the moving member past the detent 63.

The rod 65 linearly reciprocates according to movement of the moving member 61. The rod 65 is inserted through a bearing 66 disposed on the distal end side of the stopping member 63. The bearing 66 has a region made of resin, on which the rod 65 slides. The operating wire 68 has a first end on the proximal end side connected to the rod 65 and a second end on the distal end side connected to the raising base 55 that is the actuating mechanism. In other words, the operating wire 68 is inserted through the insertion portion 10.

The rod 65 has a proximal end and a distal end, the proximal end of the rod 65 is connected to the moving member 61 and the rod 65 is translatable in conjunction with the moving member 61. The wire 68 is attached to the distal end of the rod 65, the wire 68 extends into the insertion portion 10 and is attached to the actuating mechanism, the wire 68 is movable in conjunction with the rod 65 to operate the actuating mechanism. The bearing 66 is located distally relative to the moving member 61, the bearing 66 includes an opening having a contact surface. The rod 65 extends through the opening and is in slidable contact with the contact surface, and the contact surface is made of resin. The wire 68 may be indirectly attached to the distal end of the rod 65. The wire 68 may be indirectly attached to the actuating mechanism. The wire 68 may be formed of a single element or several elements. The bearing 66 may be integrated with the detent 63.

The operating wire 68 is inserted through the wire pipe 69. The proximal end side of the wire pipe 69 and the rod 65 are disposed inside the tubular member 70. Note that, in FIG. 5 and the like, the rod 65 is not disposed inside the tubular member 70. However, the rod 65 is disposed inside the tubular member 70 when the moving member 61 moves to the position indicated by the dashed line.

The wire pipe 69 contains a part of the wire 68. The tubular member 70 includes a conduit having an interior surface defining a first interior volume. At least a portion of the tubular member 70 is located between the bearing 66 and the wire pipe 69, and the wire 68 extends through the first interior volume of the tubular member 70. The tubular member 70 can include the holding member 71, the first ring receiver 72. One or more of the tubular member 70, the holding member 71 and the first ring receiver 72 can be formed in one structure. The bearing 66 may be integrated with the tubular member 70 meaning that those two features are in one structure.

The holding member 71 is fixed to the proximal end side of the tubular member 70 with an adhesive, solder, or the like, and integrated with the tubular member 70. The holding member 71 and the tubular member 70 are made of metal.

The tubular member 70 has a first section with a first inner diameter and a second section with a second inner diameter. The first inner diameter is larger than the second inner diameter, and the first section is located distally relative to the second section. The second section can be located at the holding member 71 included in the tubular member 71. The tubular member 70 may be integrated with the housing 71.

As shown in FIG. 6, the holding member 71 has an elongated through hole H71A that allows insertion of the rod 65, and a flow path H71B that communicates with the through hole H71A and has an opening on the outer surface of the holding member 71. As shown in FIG. 4, the flow path H71B connects to the flow path port 71B on the outer surface of the operation portion 20 through a tube 71A. The flow path port 71B may be provided at another position of the operation portion 20.

The operation portion 20 is disposed on a proximal end side of the insertion portion 10, the operation portion 20 includes the operating lever 24 movable between a first position and a second position.

The first ring receiver 72 is fixed to one of base members (base housing) 20A. The first ring receiver 72 has a through hole to which the wire pipe 69 is fixed. The first resin ring 73 is placed in a gap between the first ring receiver 72 and an inner peripheral surface 70SB on the distal end side of the tubular member 70.

Note that, as shown in FIG. 6 and so on, the inner diameter of the distal end side of the tubular member 70 that allows insertion of the first ring receiver 72 and the first resin ring 73 is larger than the inner diameter of the proximal end side to which the holding member 71 is joined. The tubular member 70 functions as a ring receiver on which the first resin ring 73 is placed.

The first sealing ring 73 is provided around the wire pipe 69 and contacts the interior surface to form a first seal at a distal end of the conduit. The first sealing ring 73 is made of a compliant material, such as silicone rubber, nitrile rubber, fluoro rubber, urethane rubber etc.

The second resin ring 74 is placed in a gap between an outer peripheral surface 65SA of the rod 65 and an inner peripheral surface 71SB on the proximal end side of the holding member 71. The holding member 71 has the function of a ring receiver on which the second resin ring 74 is placed.

The holding member 71 is connected to a proximal end of the tubular member 70 and has a second interior volume. The second sealing ring 74 is provided around the rod 65 and contacting an interior surface of the holding member 71 to form a second seal. The second sealing ring 74 is made of a compliant material, such as silicone rubber, nitrile rubber, fluoro rubber, urethane rubber etc. The first sealing ring 73 is formed of a first resin and the second sealing 74 ring is formed of a second resin. The housing 71 includes an opening between the second interior volume and an exterior surface of the housing 71. The second interior volume is in fluid communication with the first interior volume, and the opening is configured for injection of a sterilizing gas into the first interior volume and the second interior volume.

In the endoscope 1 of the embodiment, the first resin ring 73 and the second resin ring 74 are O-shaped or C-shaped rings that may be configured to seal the gap in a watertight manner. Therefore, the inside of the tubular member 70 between the first resin ring 73 and the second resin ring 74 is sealed from the outside of the tubular member 70.

Before shipping, endoscopes are sterilized with, for example, ethylene oxide gas. For example, a chamber of a sterilizing apparatus (not shown) is decompressed after the endoscope is accommodated. A sterilizing gas is then injected into the chamber. The sterilizing gas is injected into the tubular member 70 through the flow path port 71B of the operation portion 20 and the tube 71A. The sterilizing gas also reaches the gap between the wire pipe 69 and the operating wire 68. In the endoscope 1, the space into which the sterilizing gas is injected is sealed. Therefore, the sterilizing gas does not adversely affect electronic components and the like placed in the internal space of the operation portion 20.

While the endoscope 1 is in use, body fluid may flow back into the wire pipe 69 through the opening H50B of the distal end portion 11. In the case of a reusable endoscope, disinfection/sterilization processing with a cleaning liquid is performed after use. Since the internal space of the endoscope 1 is sealed, the cleaning liquid does not adversely affect the electronic components and the like placed in the internal space of the operation portion 20.

The transmission mechanism 60 is placed on a plurality of base members 20A fixed to the operation portion 20. The wire pipe 69, the first ring receiver 72, the holding member 71, the bearing 66, and the stopping member 63 are disposed and fixed to the base members 20A in the order described above. The operation portion 20 may include a base housing 20A. The tubular member 70 may be connected to the base housing 20A to prevent a movement of the tubular member 70 relative to the base member 20A.

The raising base 55, the operating wire 68, the rod 65, the moving member 61, and the operating lever 24 are movable members disposed from the distal end side to the proximal end side in the order described above.

### Operation of Transmission Mechanism

As indicated by the dashed line in FIG. 4, when the operating lever 24 is rotated around a central axis A24, the moving member 61 connected to the link rod 26 linearly moves toward the distal end side (in a Z-axis direction). In other words, since both side surfaces 61SS of the moving member 61 are sandwiched between the rail members 79, rotational motion of the operating lever 24 is converted into linear motion by a link mechanism. As already described, when the moving member 61 comes into contact with the stopping member 63, the moving member 61 stops at the position indicated by the dashed line in FIG. 5.

Movement of the moving member 61 to the distal end side moves the rod 65 to the distal end side, causes the operating wire 68 to press the raising base 55, and causes the raising base 55 to be into the lying state. The rotation of the operating lever 24 in a direction opposite to the above moves the moving member 61 to the proximal end side, causes the operating wire 68 to pull the raising base 55, and causes the raising base 55 to be in a rising state.

In a conventional endoscope, such as an endoscope disclosed in Japanese Patent Application Laid-Open Publication No. 2020-89598, the holding member 71 has served also as a bearing for the rod 65. In other words, the inner diameter of the through hole H71A that allows insertion of the rod 65 of the holding member 71 is set slightly larger than the outer diameter of the rod 65. Since the inner peripheral surface of the through hole H71A of the metal holding member 71 slides on the outer peripheral surface of the rod 65, a large frictional resistance is generated when the rod 65 moves. Therefore, there has been a problem such that uncomfortable feeling and rattling during operation makes it difficult to perform accurate operation.

In contrast, the endoscope 1 of the present embodiment includes the rod 65 having the bearing 66 that is entirely made of polyacetal resin (POM). POM is hard and has low sliding resistance with metal. At least the sliding surface of the bearing 66 may be made of resin having high mechanical strength and self-lubricating properties, such as POM, polyamide, polycarbonate, modified polyphenylene ether, polybutylene terephthalate, or fluorine resin. At least either one of the outer peripheral surface of the rod 65 and the sliding surface of the bearing 66 may be roughened to reduce friction.

Note that the inner diameter of the through hole H71A of the holding member 71 is set larger than the outer diameter of the rod 65. Therefore, the rod 65 does not slide on the holding member 71. The transmission mechanism 60 does not have a metal member configured to slide on the rod 65.

Since the endoscope 1 does not generate a large frictional resistance with the bearing 66 when the rod 65 moves, the operation with the operating lever 24 is comfortable and accurate operation is easy.

### Configuration of Fixing Member

As shown in FIG. 6, the bearing 66 is fixed to the proximal end side of the holding member 71 with a nut 67. The nut 67 covers the periphery of a proximal end side opening of the bearing 66 and is screwed onto the holding member 71. The nut 67 can fix the bearing 66 to the tubular member 70.

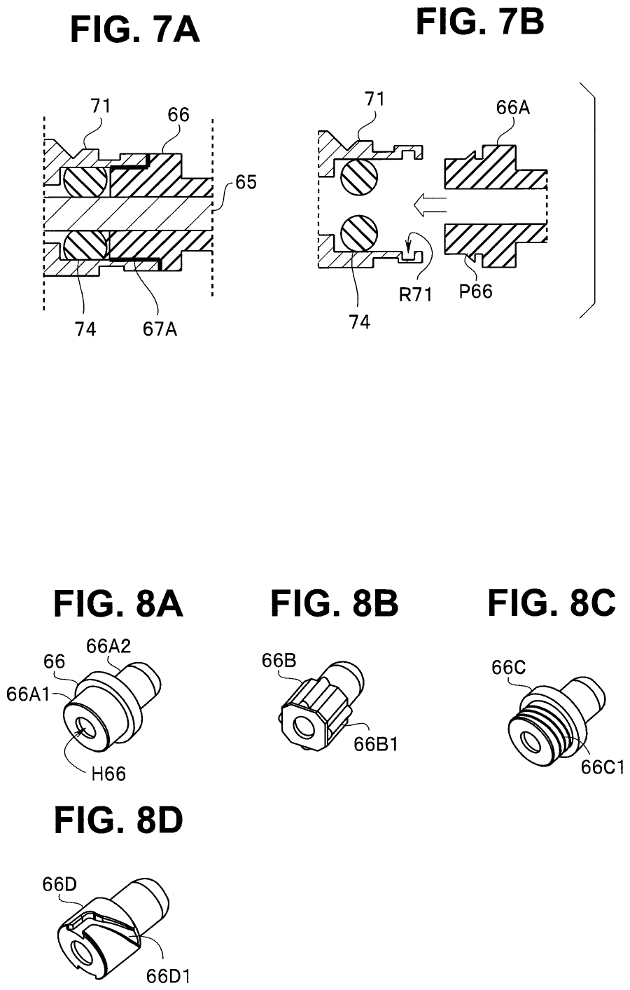

As in a modification shown in FIG. 7A, the bearing 66 may be fixed to the holding member 71 with an adhesive 67A. Further, as in a modification shown in FIG. 7B, a bearing 66A having a protrusion P66 on the outer periphery may fit into a holding member 71 having a recess R71 on the inner surface. Alternatively, the bearing 66A may be press-fitted into the holding member 71, as is to be described below.

### Configuration of Bearing

As shown in FIG. 8A, the bearing 66 has: a cylindrical distal end portion 66A1 to be inserted into the holding member 71; a cylindrical proximal end portion 66A2 to be inserted into the stopping member 63; and a through hole H66 having a circular cross section that allows insertion of the rod 65. The shape of the bearing and the method of fixing the bearing and the holding member 71 are not limited to the embodiment.

A bearing 66B in a modification shown in FIG. 8B has a distal end portion, to be inserted into the holding member 71, which has a substantially rectangular parallelepiped shape with a press-fit projection 66B1 on the outer surface. A bearing 8C in a modification shown in FIG. 8C has a thread groove 66C1 formed in a cylindrical distal end portion to be inserted into the holding member 71. The distal end portion of the bearing 8C is screwed into a screw hole of the holding member 71. A bearing 66D in a modification shown in FIG. 8D has a fitting groove 66D1 formed in a cylindrical distal end portion. The bearing 8D is fixed to the holding member 71 with the fitting groove 66D1 fitting on a projection (not shown) of the holding member 71.

The bearing 66 includes feature engaging with the tubular member 70, the feature is a protrusion, a recess, or a thread of a threaded connection.

### Configuration of Sliding Portion

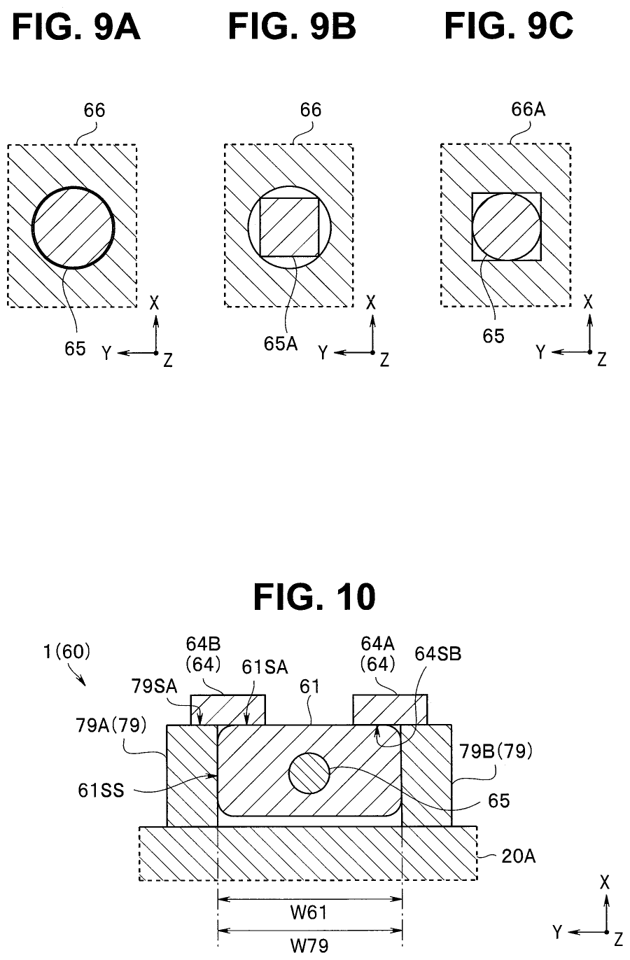

As shown in FIG. 9A, the outer peripheral surface of the cylindrical rod 65 and the sliding surface of the inner peripheral surface of the bearing 66 are in surface contact.

In contrast, a rod 65A in a modification shown in FIG. 9B is a prism, so that the sliding surface with the inner peripheral surface of the bearing 66 is in line contact. Similarly, a bearing 66A in a modification shown in FIG. 9C has a hole with a square cross section, so that the cylindrical rod 65 and the inner peripheral surface of the bearing 66 are in line contact. The rod 65 may be polygonal such as a hexagon, and the cross section of the hole of the bearing 66 may also be polygonal as long as the hole has a shape that makes line contact with the rod 65.

The opening of the bearing 66 has a first shape, a cross-section of the rod 65 has a second shape. The first shape may be same as the second shape. The first shape may be different from the second shape, and the rod 65 may contact a part of the bearing 66.

A resin ring can be located in a gap between the opening and the rod 65. The resin ring can form a seal between the bearing 66 and the rod 65.

A sliding surface in line contact has less frictional resistance than a sliding surface in surface contact.

### Configuration of Moving Member

As shown in FIG. 10, both side surfaces 61SS of the substantially rectangular parallelepiped moving member 61 are sandwiched between two rail members 79 (79A and 79B) disposed parallel to the moving direction (the Z-axis direction in the drawing). In other words, an interval W79 between the rail members 79 is set slightly larger than a width W61 of the moving member 61 (the interval between the two side surfaces 61SS).

A surface of the moving member 61 includes the protrusion 62 having the opposite side surfaces 61SS separated by a distance defining an outer width. The guide member 79 includes two surfaces separated by a distance defining an inner width W79, the inner width is less than the outer width.

A link mechanism applies stress to the moving member 61 in directions other than the moving direction (the Z-axis direction in the drawing). To prevent the moving member 61 from moving in an upper direction (X-axis direction in the drawing) perpendicular to the moving direction, guide plate 64 extends from the stopping member 63 in a proximal direction.

For example, similarly to the endoscope disclosed in Japanese Patent Application Laid-Open Publication No. 2020-89598, the guide plate 64 has a lower surface 64SB in contact with the upper surface 61SA of the moving member 61. The guide plate 64 includes a pair of elongated guide plates 64A and 64B that look like a bird with the wings spread so that the guide plate 64 is in contact with the upper surface 61SA of the moving member 61 while the guide plate 64 avoids screws (not shown) provided on the upper surface 61SA. The guide member 64 can include the guide plate 64, or the guide member 64 and the guide plate 64 can be formed in one structure.

### Second Embodiment

An endoscope 1A of a second embodiment is similar to the endoscope 1 of the first embodiment and has the same functions. Therefore, in the following description, components having the same functions as the components of the endoscope 1 are given the same reference numerals and characters, and descriptions of the components are omitted.

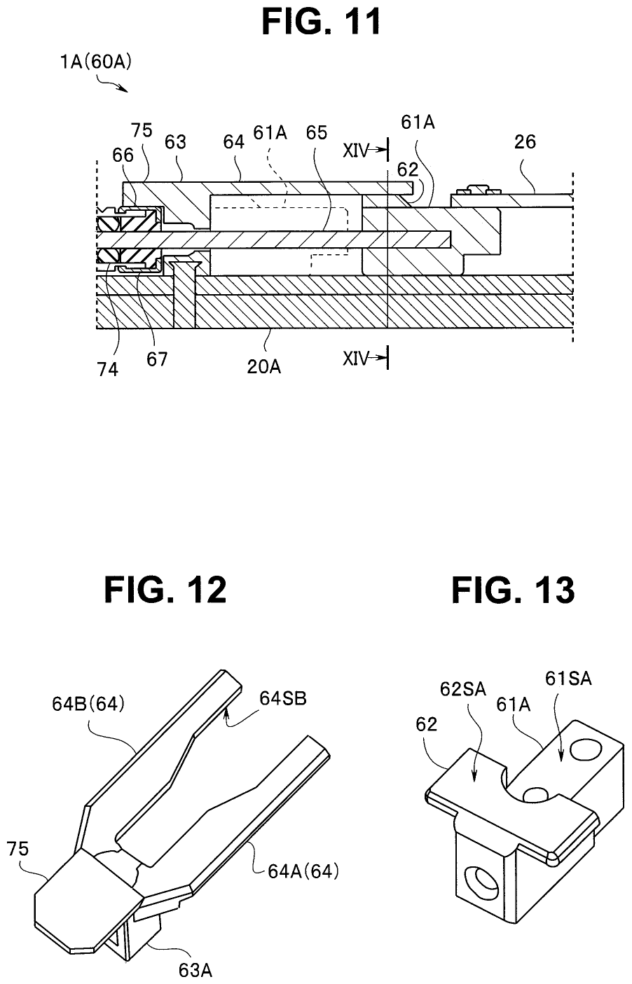

As shown in FIGS. 11 and 12, the stopping member 63A of the endoscope 1A has a first fixing plate 75 extending in a distal end direction in addition to the guide plate 64 (64A and 64B) extending in a proximal end direction. Further, as shown in FIGS. 11 and 13, in the endoscope 1A, the moving member 61A has a guided member (protrusion) 62 on the upper surface 61SA.

The first fixing plate 75 fixes the upper surface (fixing member 67) of the bearing 66 to one of the base members 20A. Therefore, the bearing 66 does not move in the upper direction.

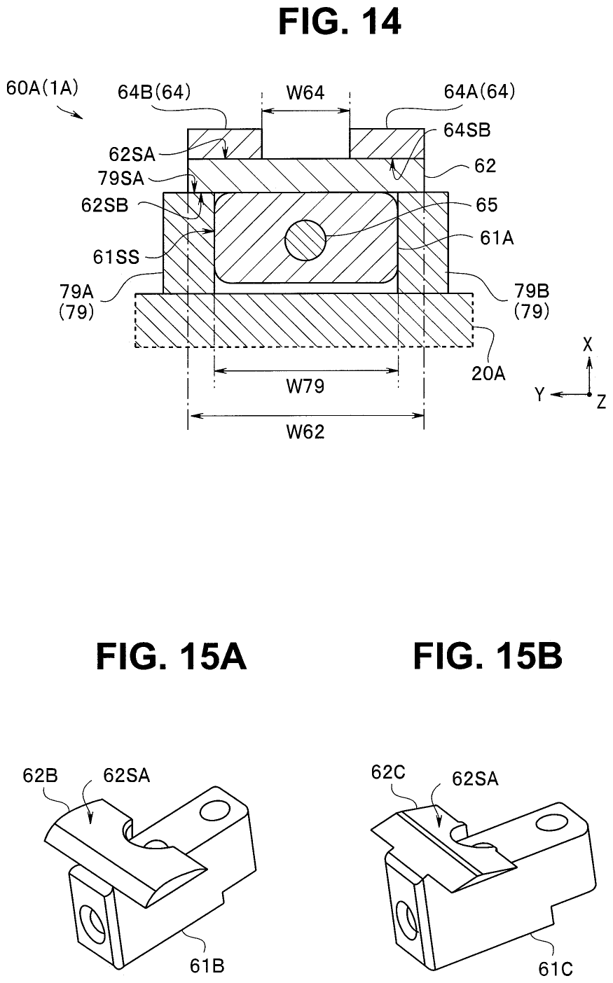

As shown in FIG. 14, the lower surface 64SB of the guide plate 64 is in contact with an upper surface 62SA of the guided member 62 rather than the upper surface 61SA of the moving member 61. A width W62 of the guided member 62 is wider than the width W61 of the moving member 61A. The contact area between the guide plate 64 and the guided member 62 is larger than the contact area between the guide plate 64 and the upper surface of the moving member 61A. Therefore, the moving member 61A having the guided member 62 moves more stably than the moving member 61 not having the guided member 62.

A guided member 62B of a moving member 61B in a modification shown in FIG. 15A has a curved upper surface 62SA. A guided member 62C of a moving member 61C in a modification shown in FIG. 15B has an upper surface 62SA having a substantially triangular cross section. The moving members 61B and 61C of the modification each have a small contact area with the guide plate 64, so that the friction is small.

The surface of the moving member 61 includes the protrusion 62 having the opposite side surfaces 61SS separated by a distance defining the outer width W62. The guide member 79 includes the two surfaces separated by a distance defining an inner width W79, the inner width is less than the outer width. The guide member 79 includes a space in which a portion of the protrusion 62 is located, and edges surfaces 64 of the space are located to prevent the moving member 61 from separating from the guide member 79 during translation of the moving member 61.

### Third Embodiment

An endoscope 1B of a third embodiment is similar to the endoscopes 1 and 1A and has the same functions. Therefore, in the following description, components having the same functions as the components of the endoscope 1 are given the same reference numerals and characters, and descriptions of the components are omitted.

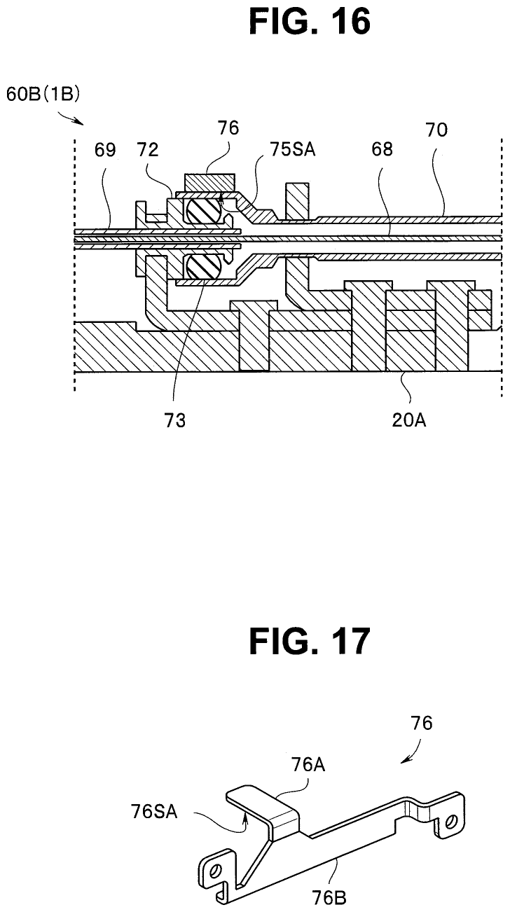

As shown in FIG. 16, the endoscope 1B has a second fixing plate 76 configured to fix an upper surface 75SA of a distal end side region of the tubular member 70 where the first resin ring 73 is placed.

As shown in FIG. 17, the second fixing plate 76 has a fixing plate 76A having a contact surface 76SA to be in contact with the tubular member 70 and a fixing plate 76B to be fixed to one or more of the base members 20A.

The second fixing plate 76 restricts movement of the distal end of the tubular member 70 in the upper direction. Therefore, the endoscope 1B has good operability.

### Fourth Embodiment

An endoscope 1C of a fourth embodiment is similar to the endoscopes 1, 1A and 1B and has the same functions. Therefore, in the following description, components having the same functions as the components of the endoscope 1 are given the same reference numerals and characters, and descriptions of the components are omitted.

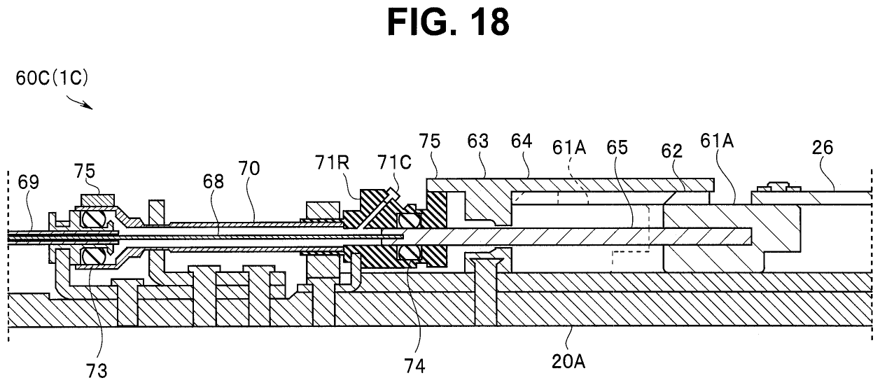

As shown in FIG. 18, the endoscope 1C has a holding member 71R made of resin that also serves as a bearing. The holding member 71R is made of resin, having high mechanical strength and self-lubricating properties, such as POM, polyamide, polycarbonate, modified polyphenylene ether, polybutylene terephthalate, or fluorine resin.

The endoscope 1C is easier to manufacture than the endoscopes 1, 1A, and 1B because the components of a transmission mechanism 60B are fewer.

The actuating mechanism is not limited to the raising base 55, and may be, for example, a zoom mechanism of an image pickup optical system or a rigidity changing mechanism of the insertion portion.

In the above embodiment, the endoscope 1 is a single-use endoscope to be disposed of after being used once, but may be a re-use endoscope to be repeatedly used, but may be a reusable endoscope that is used repeatedly. The endoscope 1 is a flexible endoscope having the insertion portion 10 with a flexibility, but may be a rigid endoscope having a rigid straight tube as the insertion portion. Furthermore, the endoscope 1 and the like are not limited to medical use, and may be industrial use.

The present disclosure is not limited to the embodiment explained above. Various changes, alterations, and the like are possible within a range not changing the gist of the disclosure.

Example 1. An endoscope comprising:
an insertion portion inserted into a subject, the insertion portion including an actuating mechanism at a distal end portion;
an operation portion disposed on a proximal end side of the insertion portion, the operation portion including an operating lever; and
a transmission mechanism configured to transmit operation of the operating lever to the actuating mechanism,
wherein the transmission mechanism includes:
   a moving member configured to reciprocate linearly according to operation of the operating lever;
   two rail members sandwiching two side surfaces of the moving member, the side surfaces being parallel to a moving direction of the moving member;
   a stopping member configured to restrict a moving range of the moving member;
   a metal rod connected to the moving member;
   a bearing disposed on a distal end side of the stopping member, the bearing allowing insertion of the rod, the bearing including a region on which the rod slides, the region being made of resin; and
   an operating wire inserted through the insertion portion, the operating wire including a first end connected to the rod and a second end connected to the actuating mechanism.

Example 2. The endoscope according to Example 1, wherein
the transmission mechanism further includes:
   a wire pipe allowing insertion of the operating wire;
   a tubular member inside which a proximal end side of the wire pipe and the rod are disposed;
   a holding member fixed to a proximal end side of the tubular member, the holding member including a through hole and a flow path, the through hole allowing insertion of the rod, the flow path being connected to the through hole, the flow path including an opening on an outer surface of the flow path;
   a first resin ring placed in a gap between a first ring receiver and an inner peripheral surface of the tubular member on a distal end side, the first ring receiver being a receiver to which the wire pipe is fixed; and
   a second resin ring placed in a gap between an outer peripheral surface of the rod and an inner peripheral surface of the holding member on a proximal end side, and
an inside of the tubular member between the first resin ring and the second resin ring is sealed against an outside of the tubular member in a watertight manner.

Example 3. The endoscope according to Example 1, wherein
the bearing is entirely made of the resin.

Example 4. The endoscope according to Example 1, wherein
the transmission mechanism does not include a metal member configured to slide on the rod.

Example 5. The endoscope according to Example 1, wherein
the bearing and the rod are in surface contact.

Example 6. The endoscope according to Example 1, wherein
the bearing and the rod are in line contact.

Example 7. The endoscope according to Example 1, wherein the moving member includes a guided member on an upper surface of the moving member, the guided member being wider than an interval between the two rail members, the endoscope further comprising
a guide plate in contact with the guided member.

Example 8. The endoscope according to Example 7, further comprising:
a first fixing plate configured to fix an upper surface of the bearing.

Example 9. The endoscope according to Example 8, wherein
the guide plate and the first fixing plate are placed on the stopping member.

Example 10. The endoscope according to Example 2, wherein
the first resin ring and the second resin ring are O-shaped rings.

Example 11. The endoscope according to Example 2, wherein
an inner diameter of the tubular member on a distal end side in which the first resin ring is placed is larger than an inner diameter of the tubular member on a proximal end side to which the holding member is joined.

Example 12. The endoscope according to Example 2, further comprising
a fixing member configured to fix the bearing to a proximal end side of the holding member.

Example 13. The endoscope according to Example 12, wherein
the fixing member is a nut that covers a periphery of an opening of the bearing and is screwed onto the holding member.

Example 14. The endoscope according to Example 2, further comprising
a second fixing plate configured to fix an upper surface of a region of the tubular member, the region being where the first resin ring is placed.

Example 15. The endoscope according to Example 2, wherein
the holding member also serves as the bearing.

Example 16. The endoscope according to Example 1, wherein
the actuating mechanism is a forceps raising base that changes a direction in which a treatment instrument protrudes from an opening of the distal end portion.

Example 17. The endoscope according to Example 1,
wherein the endoscope is a single-use endoscope that is used only once.

What is claimed is:

1. An endoscope, comprising:
an insertion portion configured for insertion into a subject, the insertion portion including an actuating mechanism;
an operation portion disposed proximally relative to the insertion portion, the operation portion including an operating lever movable between a first position and a second position;
a moving member operatively connected to the operating lever, the moving member translatable between a proximal position and a distal position in response to an operation force from the operating lever;
a detent located distally relative to the moving member, the detent configured to prevent a distally forward translation of the moving member past the detent;
a rod having a proximal end and a distal end, the proximal end of the rod connected to the moving member and the rod translatable in conjunction with the moving member;
a wire attached to the distal end of the rod, extending into the insertion portion and attached to the actuating mechanism, the wire movable in conjunction with the rod to operate the actuating mechanism; and
a bearing located distally relative to the moving member, the bearing including an opening having a contact surface,
wherein the rod extends through the opening and is in slidable contact with the contact surface, and
wherein the contact surface is made of resin.

2. The endoscope according to claim 1, further comprising:
a wire pipe containing a part of the wire; and
a tubular member including a conduit having an interior surface defining a first interior volume,
wherein at least a portion of the tubular member is located between the bearing and the wire pipe, and
wherein the wire extends through the first interior volume of the tubular member.

3. The endoscope according to claim 2, further comprising:
a first sealing ring provided around the wire pipe and contacting the interior surface to form a first seal at a distal end of the conduit.

4. The endoscope according to claim 3, further comprising:
a housing connected to a proximal end of the tubular member and having a second interior volume; and
a second sealing ring provided around the rod and contacting an interior surface of the housing to form a second seal.

5. The endoscope according to claim 4, wherein the first sealing ring is formed of a first resin and the second sealing ring is formed of a second resin.

6. The endoscope according to claim 4, wherein the housing includes an opening between the second interior volume and an exterior surface of the housing,
wherein the second interior volume is in fluid communication with the first interior volume, and
wherein the opening is configured for injection of a sterilizing gas into the first interior volume and the second interior volume.

7. The endoscope according to claim 4, wherein the housing is integrated with the tubular member.

8. The endoscope according to claim 1, further comprising:
a guide member located adjacent to the moving member;
wherein a surface of the moving member includes a protrusion having opposite side surfaces separated by a distance defining an outer width,
wherein the guide member includes two surfaces separated by a distance defining an inner width, and
wherein the inner width is less than the outer width.

9. The endoscope according to claim 8, wherein the guide member includes a space in which a portion of the protrusion is located, and wherein edges surfaces of the space are located to prevent the moving member from separating from the guide member during translation of the moving member.

10. The endoscope according to claim 4, wherein the first sealing ring and the second sealing ring are O-shaped rings.

11. The endoscope according to claim 2, wherein the tubular member has a first section with a first inner diameter and a second section with a second inner diameter,
wherein the first inner diameter is larger than the second inner diameter, and
wherein the first section is located distally relative to the second section.

12. The endoscope according to claim 2, further comprising:
a nut fixing the bearing to the tubular member.

13. The endoscope according to claim 2, wherein the operation portion includes a base housing, and
wherein the tubular member is connected to the base housing to prevent a movement of the tubular member relative to the base member.

14. The endoscope according to claim 2, wherein the bearing is integrated with the tubular member.

15. The endoscope according to claim 1, wherein the actuating mechanism is one of a forceps raising base, a zoom mechanism of an image pickup optical system, and a rigidity changing mechanism of the insertion portion.

16. The endoscope according to claim 1, wherein the endoscope is a single-use endoscope.

17. The endoscope according to claim 2, wherein the bearing includes feature engaging with the tubular member, wherein the feature is a protrusion, a recess, or a thread of a threaded connection.

18. The endoscope according to claim 1, wherein the opening of the bearing has a first shape,
wherein a cross-section of the rod has a second shape, and
wherein the first shape is different from the second shape.

19. The endoscope according to claim 18, further comprising:
a gap between the opening and the rod; and
a resin ring located in the gap,
wherein the resin ring forms a seal between the bearing and the rod.

20. The endoscope according to claim 1, wherein the bearing is integrated with the detent.
